(19) United States
(12) Patent Application Publication
(10) Pub. No.: US 2019/0175410 A1
(43) Pub. Date: Jun. 13, 2019

(54) Adjustable Goggle-Integrated Face Shield

(71) Applicant: Jeffrey Mark Eliseo, Fair Oaks, CA (US)

(72) Inventor: Jeffrey Mark Eliseo, Fair Oaks, CA (US)

(21) Appl. No.: 15/835,446

(22) Filed: Dec. 7, 2017

(51) Int. Cl. A61F 9/02

(52) U.S. Cl. CPC A61F 9/029

(57) ABSTRACT

Embodiments described herein provide for a system for a face shield typically worn while engaging in snow sports. The face shield may be worn with a pair of goggles as well as a helmet if desired. The face shield is comprised of a body portion having arms extending therefrom. One or more retainers are affixed to each of one or more adjustable brackets and one or more fixed brackets. Adjustable brackets are mounted to a sliding track permitting the face shield to be fit specifically to the user. Retainers in communication with the fixed and adjustable brackets releasably engage with the goggles when in use, effectively contouring the face shield about the face of the user.

### FIELD

The present invention generally relates to a face shield integration system for use in action sports such as skiing and snowboarding.

### BACKGROUND

Action sports such as skiing and snowboarding are becoming increasingly popular throughout the world as both a passion and form of exercise. Commonly, headgear may include goggles as a means of protecting the eyes from harmful sunny conditions as well as keeping snow and debris out of the eyes to promote visibility. Further, users may opt to wear a helmet to protect the head against impacts which are common in the high speed sports. Many other accessories may be worn, some of which promote warmth, and some are simply for achieving a desired aesthetic appearance.

Facemasks are commonly warm by skiers and snowboarders alike when conditions are cold as well as for aesthetic purposes. Primitive facemasks may simply be a handkerchief, bandana, or similar piece of cloth which can be tied about the lower face as to not cover the eyes of the user. While these facemasks help keep the user warm in hazardous conditions, they do little for protection of the face against impacts and are not integrated well with the goggle and/or helmet, causing frustration if not worn properly. Typical designs currently used are also uncomfortable and do not provide a barrier from moisture.

U.S. Pat. Pub. No. 2012/0167891 to Smaller, J discloses an adjustable face mask having a body conforming substantially to the facial structure of a wearer. The face mask is provided with a neck shield and removable filter and air apertures providing a communication of forced air to the nose and mouth of the user. The face mask can optionally provide neck protection with a descending neck member. The invention disclosed by Smaller does described a facemask conforming to the face of the user, however it does not describe integration with the goggles or helmet and as it is specifically separate from the two. Adjustability is only achieved by a hook and loop system as commonly known in the arts.

U.S. Pat. Pub. No. 2016/0029734 to Snugmug LLC discloses a facemask for use on a user's face including a substantially rigid body portion having side edges on either side thereof, arms extending rearwardly from each of the side edges of the body portion, and connectors coupled to the arms. The facemask is configured to be positioned over the bottom part of a user's face in order to cover the cheeks, chin, and/or nose of the user. Connectors are coupled to the arms in order to mount each of the arms onto a mount and to hold the body portion in position on a user's face. The connector are removable from a mount upon the application of a level of force and replaceable on the mount.

U.S. Pat. Pub. No. 2013/0014316 to Oakley, Inc. discloses a facial covering and eyewear system for improving the ease and comfort of wearing facemasks with a goggle. A facemask frame is provided that can removably attach to a facemask or face covering to a goggle. In alternate embodiments, a cloth covering can be provided among other alternative facemask devices. Interconnectivity is provided between a facemask frame and the goggle to secure the facemask relative to the goggle during use for covering a portion of the wearer's face.

While both the applications to Snugmug LLC and Oakley, Inc. provide for a removably engaged facemask, adjustable and universal connectivity is not discussed. This adjustability is necessary in the art to ensure users wearing the facemask may be comfortable with or without a helmet, as well as with a plurality of goggle brands, configurations, and face shapes. Further, neither application focuses on the facemask performing as a face shield to sufficiently protect the face from impact. The patent application to Oakley Inc. does not disclose a universal mount to any goggle on the market. Instead, the disclosure provides for a specific mounting system to be engaged as an intermediary between the face mask and the goggle.

It can be seen that an advancement in the arts surrounding face shields is needed, one such improvement is described herein.

### SUMMARY OF THE INVENTION

In embodiments of the present invention, a face shield system for adjustably attaching a face shield to a goggle is described. The face shield is comprised of a semi-rigid body portion having a central portion, and first and second arms extending laterally therefrom. The body portion has a front side and a back side. Adjustment means are positioned on the first arm and the second arm allowing for the face shield to be used by user's having any face size, as well as with users who choose to wear a helmet. One or more retainers are in communication with the adjustment means. The retainers permit the releasable engagement with a goggle such that the face shield is held on the face of the user during use.

Each retainer may be comprised of a first and second end configured to retain one another when releasably engaged with the strap of the goggle. Sliding tracks are disposed on each of the first and second arms having an adjustable bracket slidingly mounted thereon. The sliding tracks permit the user to adjust to the size and contour of their face for comfort and utility. Each adjustable bracket is comprised of front and rear components configured to receive one of the one or more retainers therebetween.

In an embodiment, one or more fixed brackets are mounted to each of the first and second arms. Each of the one or more fixed brackets is comprised of front and rear components. The front and rear components are each configured to receive one of the one or more retainers therebetween. Each retainer extends perpendicular to each arm.

In an embodiment, the first and second arms are tapered 305, 307, 309, 311 (seen in FIG. 3) from the central portion of the body, permitting adequate articulation of the user's head during athletic activities.

During user, the body portion is biased to substantially cover the user's lower face including the nose, cheeks, chin, and ears, but not the user's eyes. In embodiments, a polymeric material having optimal semi-rigid flex characteristics is utilized. The material should have similar flex characteristics and a wide range of temperatures including room-like temperatures, and near-freezing temperatures. The material of the face shield, and construction thereof should be significantly effective in protecting the user's face from impacts as well as UV radiation.

Other aspects, advantages, and novel features of the embodiments will become apparent from the following detailed description in conjunction with the figures.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a front perspective view of the face shield assembly, according to an embodiment of the present invention;

FIG. 2 illustrates a front elevation view of the face shield assembly, according to an embodiment of the present invention;

FIG. 3 illustrates a front perspective view of the face shield assembly, according to an embodiment of the present invention;

FIG. 4 illustrates a rear perspective view of the face shield assembly, according to an embodiment of the present invention;

FIG. 5 illustrates a rear elevation view of the face shield assembly, according to an embodiment of the present invention;

FIG. 6 illustrates an exploded view of the mounting system, according to an embodiment of the present invention;

FIG. 7 illustrates a left side elevation view of the face shield assembly, according to an embodiment of the present invention;

FIG. 8 illustrates a right side elevation view of the face shield assembly, according to an embodiment of the present invention;

FIG. 9 illustrates a top plan view of the face shield assembly, according to an embodiment of the present invention; and

FIG. 10 illustrates a bottom plan view of the face shield assembly, according to an embodiment of the present invention.

### DETAILED DESCRIPTION

The specific details of the single embodiment or variety of embodiments described herein are set forth in this application. Any specific details of the embodiments are used for demonstration purposes only and no unnecessary limitation or inferences are to be understood therefrom.

In general, the invention described herein relates to an integrated face shield for use in action sports including skiing and snowboarding. One may appreciate that alternate uses may be realized by those skilled in the arts, however for purposes of clarity examples used herein are limited to snow sports such as skiing and snowboarding. Further, the face shield should have a universal attachment mechanism, allowing the face shield system to releasably engage any goggle, as well as goggle/helmet configuration known in the arts.

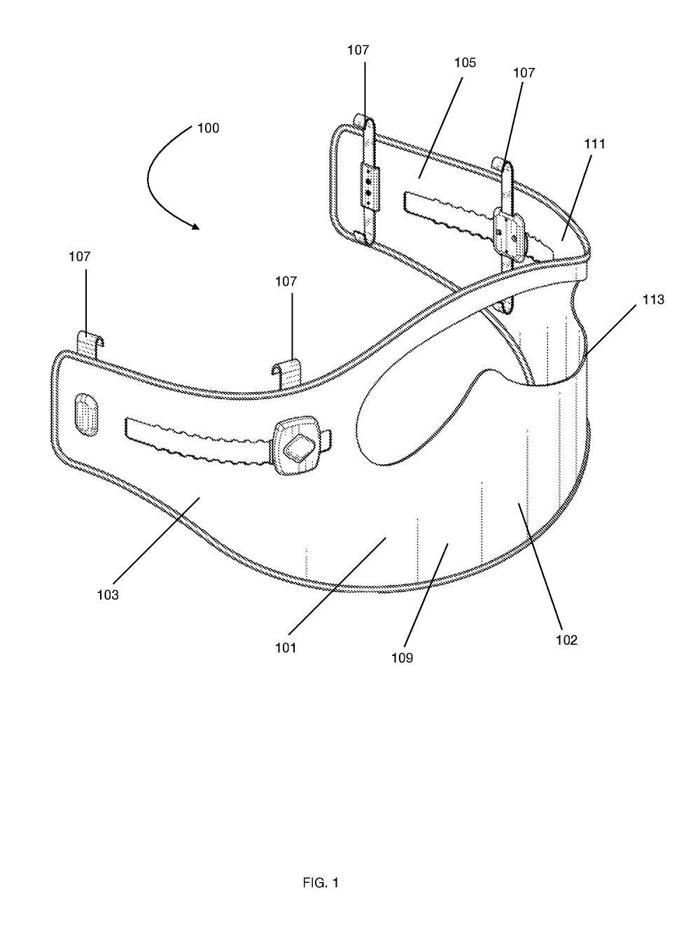

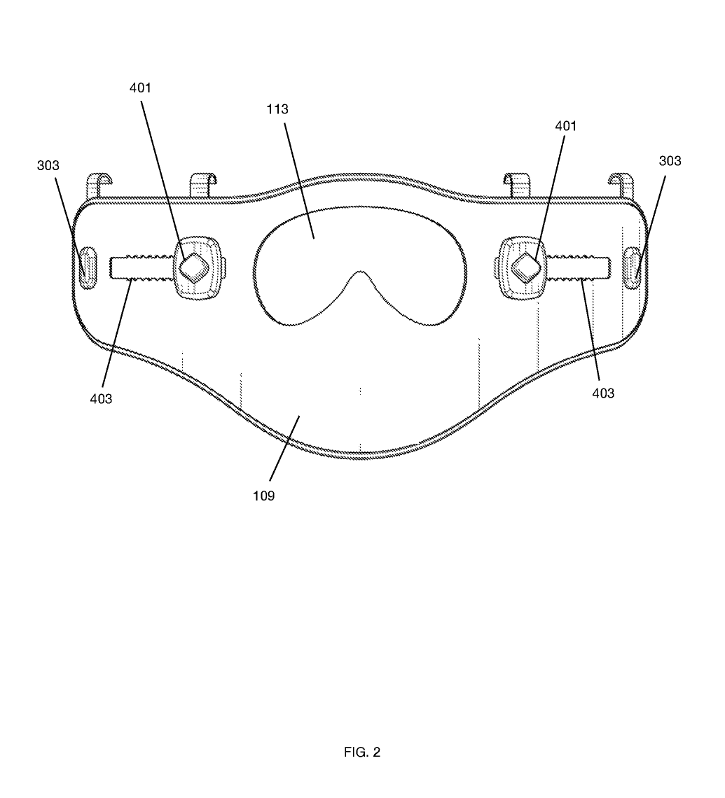

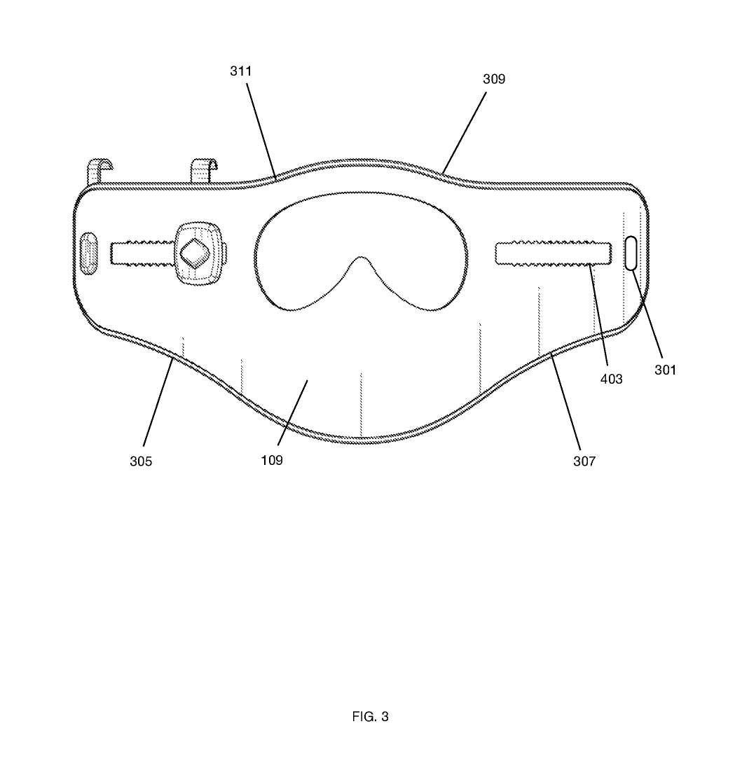

In reference to FIGS. 1-10, wherein similar components are identified by like reference numerals, FIGS. 1-3 illustrates a perspective view of the front of the device in an embodiment of the present invention. The face shield device 100 has body 101 having first and second arm portions 103, 105 extending therefrom. In a preferred embodiment, each arm 103, 105 is dimensioned to extend around the head of the user until behind each respective ear. The device 100 is selectively extendable to allow for use with or without a helmet. When a helmet is not in use, the user may selectively extend or contract the device 100 dependent on the user's specific head circumference. The body 100 has a front side 109 and a back side 111 wherein the back side 111 faces the user, while the front side 109 opposes the back side 111, and thus faces away from the user.

In a preferred embodiment, each arm 103, 105 has a plurality of apertures disposed therethrough such as to mount brackets thereon. Specifically, one or more distal apertures 301 are disposed through each arm to facilitate the mounting of a fixed bracket 303. Further, a set of medial apertures are positioned medial in relation to the distal apertures 301 permitting the engagement of an adjustable bracket 401 on each. Each adjustable bracket may slide along its respective medial aperture, such that the medial aperture is configured as a sliding track 403. The sliding engagement between the adjustable bracket 401 and sliding track 403 permit the user to modify the configuration of the device 100 to fit varying facial, helmet, and goggle configurations as well as combinations thereof.

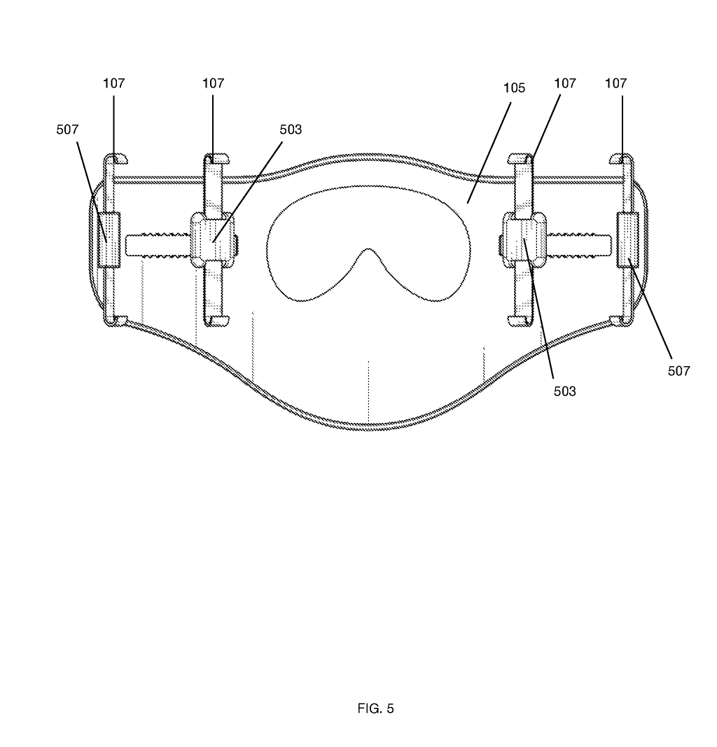

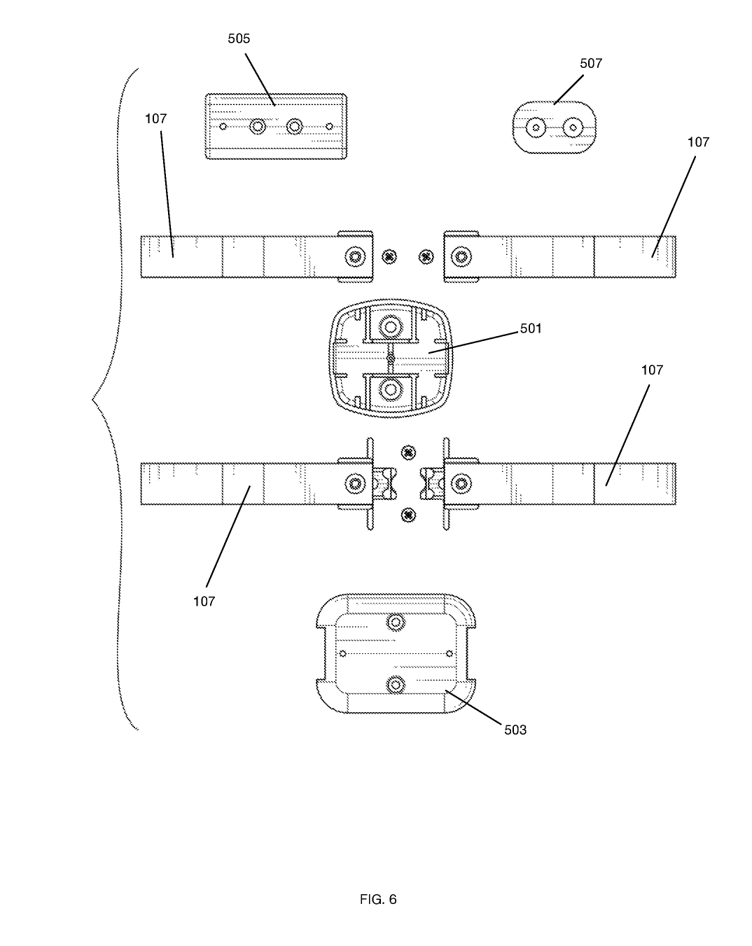

In reference to FIG. 5 and FIG. 6 each adjustable bracket 401 is comprised of a front component 501 positioned on the front side 109 of the body 101, and a back component 503 positioned on the back side 111 of the body 101 having one or more means for connecting each such as screws, pins, lockers, or other means known in the arts. Similarly, each fixed bracket is comprised of a front component 505 and back component 507, being coupled by one or more means for connecting each such as screws, pins, lockers, or other means known in the arts.

In a preferred embodiment, the fixed bracket 303 front component 501 and back component 503 are configured, when connected, to receive a retainer 107 therethrough. Similarly the adjustable bracket 401 having engaged front and back components 505, 507, is configured to receive a retainer 107 therethrough.

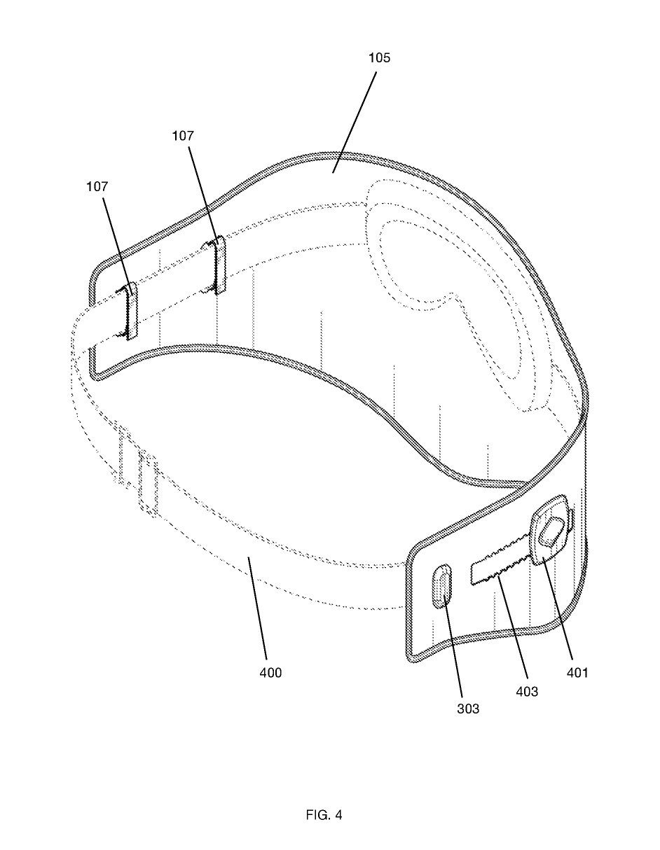

In specific reference to FIG. 4 and FIG. 5, the plurality of retainers 107 are configured to releasably engage the face shield device 100 to any goggle apparatus available. Specifically, the retainers are configured to releasably engage with a strap present in any goggle apparatus. In an embodiment, each of the plurality of retainers 107 may be affixed, or releasably engaged with the back side 111 of the body 101 my any means of textile adhesion or releasable adhesion known in the arts including sewing, integral molding, hook and loop components, among others. Each of the plurality of retainers 107 has a first and second ends 113, 115, each of which extends perpendicular to the arms 103, 105 and is dimensioned to be self retained when extending about the goggle strap 400 by hook and loop components, or similar mechanisms known in the arts.

While prior art describes goggle integration at the lens frame using intermediary connection components, the present invention releasably integrates the face shield with the goggle strap 400. This transfers the weight of the face shield from the front of the users face—as seen in prior art—to the side of the head. This transfer of weight allows for a much more convenient connection means, as well as added comfort as the user's head and neck are not strained and pulled forward by the weight of the face shield.

In a preferred embodiment, retainers engaged to adjustable brackets 401 may slide along the track as permitted by the user to achieve a proper fit.

The body of the device is sufficiently rigid to protect the face against impacts from object, such as when moving through tree branches, or similar hazards. While rigid enough to withstand the described impacts, the device 100 is biased toward the contour of the user's face when in use and connected with the goggles, as seen in FIG. 4.

Specifically referring to the retainers 107, and in reference to FIG. 5, each arm 103, 105 may have a set of retainers including first retainer set 207 and second retainer set 209. Each retainer is fixedly or releasably engaged with the device 100 utilizing adjustable brackets 401. Each adjustable bracket 401 is engaged with a track, permitting the brackets 401 to slide along the track and thus adjusting the fit of the device 100 to the user's face and goggle configurations.

In a preferred embodiment and in reference to FIGS. 1-5, one or more sliding tracks are disposed on each arm 103, 105 of the face shield. Sliding tracks have at least one adjustable bracket 401 slidingly mounted thereon. Adjustable brackets may have a spring-like engagement to bias the brackets in a direction along the sliding track such that a secure fit is achieved.

In specific reference to FIGS. 1-4, the face shield device 100 has a goggle aperture 113 configured to permit goggles 400 to be disposed therein. Goggles 400 may be seamlessly integrated into the form of the face shield device, such that no part of the users face is exposed. In further embodiments, a general goggle aperture 113 shape may be utilized to allow for goggles of varying sizes to be utilized with the invention.

The body 101 and arms 103, 105 are dimensioned to sufficiently cover the lower face of the user without obstructing or prohibiting the user from wearing goggles 400 with or without the use of a helmet. The device 100 is dimensioned to continuously cover the face below the goggles such that when worn with a pair of goggles 400, the entire face of the user is covered when the device 100 is in use. In a preferred embodiment, the device 100 protects below the chin of the user extending to a point on the user's neck allowing for maximum natural articulation of the head such that the user may look in all directions as normally able without significant restriction.

In a preferred embodiment, to aid in comfort and utility, arm portions 103, 105 of the face shield device 100 may be tapered from the central portion 102 of the body portion 101. The tapered portions 104 permit unobstructed movement of the user.

In order for the device to be sufficiently rigid, while maintaining the ability to be biased against the contour of the user's face, the body 101 and arms 103, 105 can comprise any of a variety of metals, composites, or relatively-rigid molded thermoplastics as known in the arts and combinations thereof in any number of layers. These materials may be substantially transparent, translucent, or opaque, as well as a variety of colors, patterns, and combinations thereof. The flexibility of the material permits a customized fit to the user's face and may adapt to a plurality of goggle, helmet, and head sizes and configurations. The user may be an adult or child as the modularity of the device 100 permits any number of facial sized and structures to be accommodated. The device may be manufactured by injection molding, machining, and/or other construction techniques well known in the arts.

In a preferred embodiment, the face shield assembly 100 provides significant ultra-violet ray (UV) protection as a result of the material utilized in its construction. Significant UV protection may be defined by principles well known in the arts.

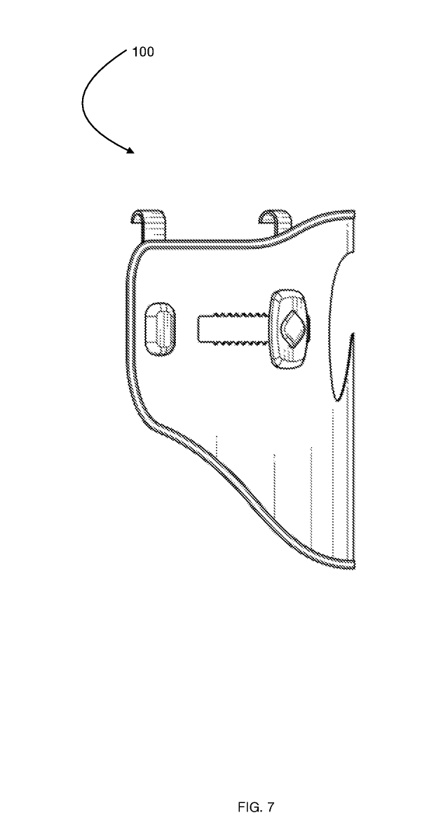

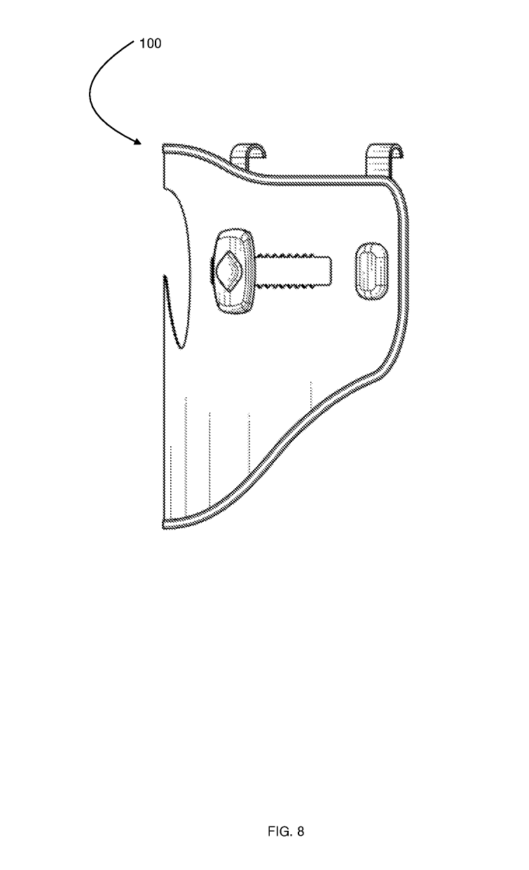

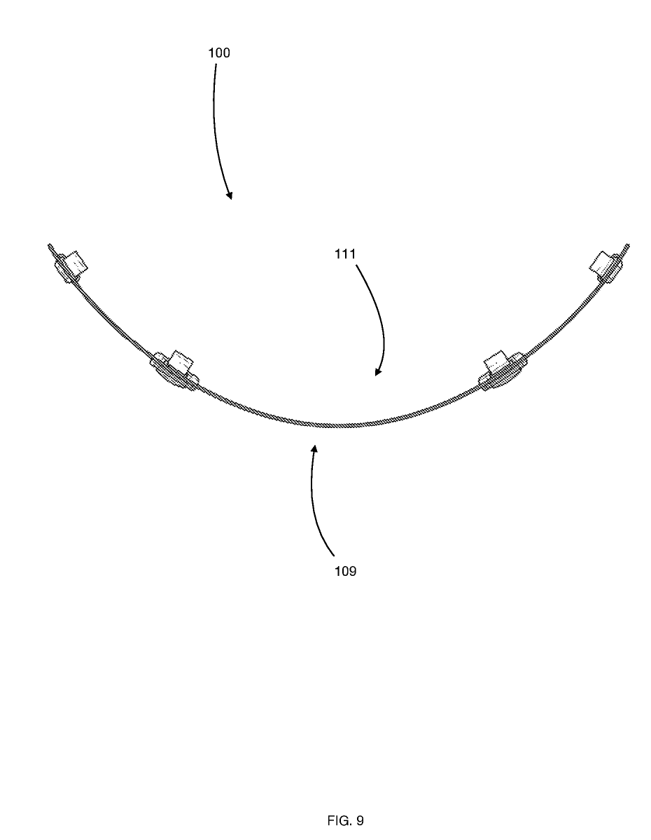

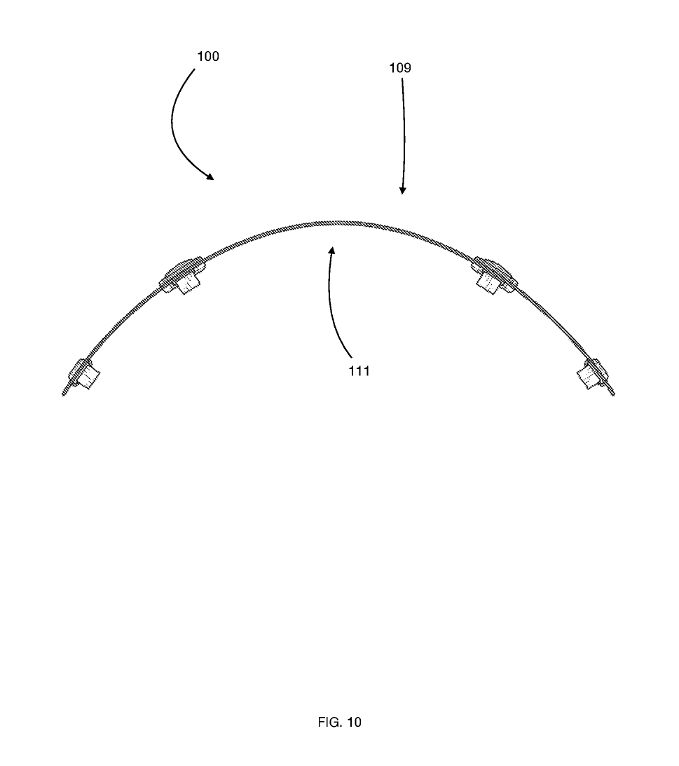

FIGS. 7-8 illustrate left and right side elevation views of the device, in an embodiment of the present invention. Further, FIGS. 9-10 illustrate top and bottom plan views of the face shield, in an embodiment of the present invention. FIGS. 7-10 specifically illustrate the biased configuration of the face shield, when in an in-use configuration.

As the device is constructed to be used in snow sports wherein climates are commonly at or below freezing temperatures, the material of the device 100 should maintain the above described rigid, yet flexible nature at a wide range of temperatures commonly encountered during snow sports. Such materials may be readily determined by those skilled in the arts of thermoplastics and composites among other like materials. Near freezing may be defined as any temperature significantly below room temperature, and more specifically any temperature under 40° F., while room temperature may be defined as any temperature above 40° F.

The body portion may be described as a semi-rigid construction permitting the face shield to bias toward the contour of the users face when in use.

In a preferred embodiment, the face shield is releasably engaged with the goggles (see FIG. 4) utilizing the goggle strap. Compared with prior art, the face shield assembly is constructed to have a universal fit with any goggle configuration currently on the market. Typically, goggles, have a two inch strap securing the goggle to the face of the user. As seen in FIG. 4, each retainer engages with the strap without the use of an intermediary connection mechanism. To releasably engage the device 100 with a goggle strap 400, no additional components are needed other than the retainers 107 and alternative embodiments thereof.

It will be appreciated by persons skilled in the art that the present embodiment is not limited to what has been particularly shown and described hereinabove. A variety of modifications and variations are possible in light of the above teachings without departing from the following claims.

What is claimed is:

1. A face shield system for attaching a face shield to a goggle comprising;
a. a semi-rigid body portion having a central portion, and having a first arm and a second arm extending laterally from the central portion, wherein the body portion has a front side and a back side;
b. one or more adjustment means positioned on the first arm and the second arm; and
c. one or more retainers in communication with the adjustment means, wherein each of the one or more retainers releasably engage a goggle, wherein each of the one or more retainers are configured to hold the face shield in position on a user's face.

2. The system of claim 1, wherein each of the one or more retainers is comprised of a first end and a second end, wherein the first and second ends are configured to releasably engage one another when retaining a goggle strap.

3. The system of claim 1, having one or more sliding tracks disposed on each of the first arm and the second arm.

4. The system of claim 3, wherein an adjustable bracket is slidingly engaged with each of the one or more sliding tracks.

5. The system of claim 4, wherein the adjustable bracket is comprised of a front component and a rear component, wherein one of the one or more retainers is disposed therebetween.

6. The system of claim 1, having one or more fixed brackets mounted to each of the first arm and the second arm.

7. The system of claim 1, wherein each of the one or more fixed brackets is comprised of a front component and a rear component, wherein the front and rear components are each configured to receive one of the one or more retainers therebetween.

8. The system of claim 1, wherein each of the first and second arms are tapered from the central portion.

9. The system of claim 1, wherein the body portion biased to substantially cover the user's lower face including the nose, cheeks, chin, and ears, but not the user's eyes.

10. The system of claim 8, wherein the adjustments means permits the face shield system to be utilized with a helmet.

11. The system of claim 8, wherein the adjustment means permits a range of face sizes to be effectively covered and biased about the face of the user.

12. The system of claim 1, wherein the body portion is constructed of a polymeric material.

13. The system of claim 1, wherein the polymeric material has substantially similar flexible characteristics near room-like temperatures and near freezing temperatures.

14. The system of claim 1, wherein the face shield significantly protects the user's face from impacts.

15. The system of claim 1, wherein the face shield is configured to provide UV protection to the user.
